(19) United States
(12) Patent Application Publication
(10) Pub. No.: US 2025/0186758 A1
(43) Pub. Date: Jun. 12, 2025

(54) CIRCULATORY SUPPORT DEVICE WITH DRUG ELUTING COATING

(71) Applicant: Boston Scientific Scimed, Inc., Maple Grove, MN (US)

(72) Inventors: Darrin Dale Beekman, Osseo, MN (US); Steven L. Kangas, Woodbury, MN (US)

(73) Assignee: Boston Scientific Scimed, Inc., Maple Grove, MN (US)

(21) Appl. No.: 18/969,841

(22) Filed: Dec. 5, 2024

Related U.S. Application Data
(60) Provisional application No. 63/606,886, filed on Dec. 6, 2023.

(51) Int. Cl. A61M 60/216; A61M 60/81; A61M 60/90

(52) U.S. Cl. CPC A61M 60/216; A61M 60/81; A61M 60/90; A61M 2205/0216; A61M 2205/0238; A61M 2205/025; A61M 2205/04

(57) ABSTRACT

A percutaneous circulatory support device including a percutaneous blood pump coupled to a distal end region of an elongate shaft at a junction. The percutaneous blood pump includes a flexible cannula, an impeller housing, and a motor housing. The percutaneous blood pump is configured to pump blood through a housing of the percutaneous blood pump during use. A drug eluting coating is disposed on at least a portion of a surface of the percutaneous blood pump.

### CROSS-REFERENCE TO RELATED APPLICATIONS

This application claims the benefit of priority under 35 U.S.C. § 119 of U.S. Provisional Application No. 63/606,886, filed Dec. 6, 2023, the entire disclosure of which is hereby incorporated by reference.

### TECHNICAL FIELD

The present disclosure pertains to a catheter including a percutaneous blood pump. More particularly, the present disclosure pertains a percutaneous circulatory support device including a drug eluting coating.

### BACKGROUND

Percutaneous mechanical circulatory support devices, such as blood pumps can provide transient support for hours or months of use in patients whose heart function or cardiac output is compromised. The percutaneous mechanical circulatory support devices may be sufficiently flexible to be navigated through the vasculature to a patient's heart. Such devices may be navigated through the aortic arch and placed across the aortic valve, for example. Some percutaneous mechanical circulatory support devices include a purge line which can be used to infuse heparin directly to the pump to reduce thrombus risk. However, purge lines may increase the complexity of percutaneous mechanical circulatory support devices. There is an ongoing need to provide alternative medical devices as well as alternative methods for manufacturing and using medical devices. This may include the use of drug eluting coatings to reduce the risk of thrombus formation.

### BRIEF SUMMARY

This disclosure provides design, material, manufacturing method, and use alternatives for medical devices, including percutaneous circulatory support devices and associated percutaneous blood pumps.

In a first example, a percutaneous circulatory support device may comprise a flexible elongate shaft, a percutaneous blood pump coupled to a distal end region of the flexible elongate shaft at a junction, the percutaneous blood pump configured to pump blood through a housing of the percutaneous blood pump during use and a drug eluting coating disposed on at least a portion of a surface of the percutaneous blood pump.

Alternatively or additionally to any of the examples above, in another example, the drug eluting coating may comprise an excipient and a direct oral anticoagulant (DOAC).

Alternatively or additionally to any of the examples above, in another example, the excipient may comprise a thermoplastic polyurethane.

Alternatively or additionally to any of the examples above, in another example, the excipient may comprise in the range of about 60 to 95 weight percent of the drug eluting coating and the DOAC may comprise in the range of about 5 to 40 weight percent of the drug eluting coating.

Alternatively or additionally to any of the examples above, in another example, the excipient may comprise in the range of about 50 to 70 weight percent of the drug eluting coating and the DOAC may comprise in the range of about 30 to 50 weight percent of the drug eluting coating.

Alternatively or additionally to any of the examples above, in another example, the percutaneous circulatory support device may further comprise a top layer disposed over the drug eluting coating.

Alternatively or additionally to any of the examples above, in another example, the top layer may be free from a therapeutic agent.

Alternatively or additionally to any of the examples above, in another example, the DOAC may be present in the drug eluting coating in an amount of between 1-40 micrograms per square millimeter.

Alternatively or additionally to any of the examples above, in another example, the percutaneous blood pump may comprise a flexible cannula, an impeller housing, and a motor housing.

Alternatively or additionally to any of the examples above, in another example, the percutaneous circulatory support device may further comprise an impeller assembly disposed within the impeller housing

Alternatively or additionally to any of the examples above, in another example, the drug eluting coating may further comprise an additional therapeutic agent.

Alternatively or additionally to any of the examples above, in another example, the drug eluting coating may be disposed on a surface of the distal tip.

Alternatively or additionally to any of the examples above, in another example, the drug eluting coating may be disposed adjacent to the inlet windows.

Alternatively or additionally to any of the examples above, in another example, the drug eluting coating may be disposed adjacent to the sensor ports

Alternatively or additionally to any of the examples above, in another example, the flexible cannula may comprise a slotted hypotube covered with a polyurethane sheath.

Alternatively or additionally to any of the examples above, in another example, the drug eluting coating may be disposed between the slotted hypotube and the polyurethane sheath.

Alternatively or additionally to any of the examples above, in another example, the drug eluting coating may be disposed on an inner surface of the flexible cannula.

Alternatively or additionally to any of the examples above, in another example, the drug eluting coating may be disposed on a portion of the impeller housing.

Alternatively or additionally to any of the examples above, in another example, the DOAC may be apixaban, rivaroxaban, or edoxaban.

Alternatively or additionally to any of the examples above, in another example, the drug eluting coating may have a thickness in the range of about 1 to 50 micrometers.

In another example, a percutaneous circulatory support device may comprise a flexible elongate shaft, a percutaneous blood pump coupled to a distal end region of the flexible elongate shaft at a junction, the percutaneous blood pump configured to pump blood through a housing of the percutaneous blood pump during use, and a drug eluting coating disposed on at least a portion of a surface of the percutaneous blood pump. The drug eluting coating may comprise an excipient and an anticoagulant.

Alternatively or additionally to any of the examples above, in another example, the anticoagulant may comprise a factor Xa inhibitor.

Alternatively or additionally to any of the examples above, in another example, the excipient may comprise in the range of about 60 to 95 weight percent of the drug eluting coating and the anticoagulant may comprise in the range of about 5 to 40 weight percent of the drug eluting coating.

Alternatively or additionally to any of the examples above, in another example, the percutaneous circulatory support device may further comprise a top layer disposed over the drug eluting coating.

In another example, a percutaneous circulatory support device may comprise a flexible elongate shaft, a percutaneous blood pump including a flexible cannula, an impeller housing, and a motor housing, the percutaneous blood pump coupled to a distal end region of the flexible elongate shaft at a junction and configured to pump blood through a housing of the percutaneous blood pump during use, and a drug eluting coating disposed on at least a portion of a surface of the percutaneous blood pump. The drug eluting coating may comprise an excipient and an anticoagulant.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

The disclosure may be more completely understood in consideration of the following detailed description in connection with the accompanying drawings, in which:

FIG. 1 is a perspective view of an exemplary percutaneous circulatory support device including a percutaneous blood pump;

FIG. 2 shows the distal end region of the percutaneous circulatory support device of FIG. 1 including the percutaneous blood pump;

FIG. 3 is a side view of a portion of the percutaneous blood pump of FIG. 1;

FIG. 4 is a cross-sectional view of the portion of the percutaneous blood pump of FIG. 3;

FIG. 5 is a cross-sectional view of a portion of the percutaneous blood pump of FIG. 2; and

FIG. 6 is a graphical representation of experimental data.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

Mechanical circulatory support devices may be used to help protect patients during high risk percutaneous coronary intervention procedures, episodes of cardiogenic shock, or the like. Mechanical circulatory support devices may include a pump that transfers blood from a chamber of the heart directly to the aorta. The mechanical circulatory support device may improve systemic circulation and end-organ dysfunction among others. In some cases, the mechanical circulatory support device may “unload” the left ventricle such that the left ventricle does not have to perform as much work. The mechanical circulatory support device may remain in place for an extended period of time (e.g., in the range of hours or days). However, mechanical circulatory support devices may be used for even longer periods of time such as weeks or months. In some cases, the mechanical circulatory support device may experience thrombus formation. Some mechanical circulatory support devices may include a purge line which can be used to provide an infusion solution, which may include heparin, directly to the pump internals to reduce thrombus formation. However, the purge line may increase the complexity of the mechanical circulatory support device. Further, it may be difficult to control patient activated coagulation time (“ACT”) levels when an infusion solution is infused through the purge line. Mechanical circulatory support devices without a purge line may increase the simplicity of the system but may have an increased thrombus risk, especially for long dwell times and/or low ACT levels. The present disclosure is directed towards a mechanical circulatory support device which includes a drug eluting coating to reduce thrombogenicity. While a purge line may reduce thrombus formation, thrombus related adverse events may occur in a mechanical circulatory support device whether a purge line is present or not. It is contemplated that the drug eluting coating described herein may be used with mechanical circulatory support devices having purge lines or mechanical circulatory support devices free from purge lines.

Blood coagulation factor Xa (FXa) is a serine protease that is involved in both the blood coagulation cascade and in the expression of numerous inflammatory cytokines such as IL-6. FXa is known to activate prothrombin to thrombin which eventually leads to the formation of cross-linked blood clots. Further, FXa has also been linked to inflammation via protease-activated receptors. Drugs (such as, but not limited to, direct oral anti-coagulants (DOACs) such as rivaroxaban and apixaban) that directly bind and inhibit factor Xa are highly effective at preventing thrombus formation in patients with atrial fibrillation (A-fib) and in deep vein thrombosis (DVT) patients at reducing the risk of recurrent DVT, among other conditions.

The medical devices disclosed herein may provide direct oral anticoagulants (DOACs) on one or more surfaces of a device. The device may then be delivered to the desired treatment region. This may provide the advantage of reducing the potential adverse effects of systemic oral anticoagulant (OAC) therapy. This may be accomplished by incorporating DOACs into a polymer coating disposed on one or more portions of medical device, such as, but not limited to, a mechanical circulatory support device. While the therapeutic coating described herein is discussed relative to a mechanical circulatory support device, it is contemplated that the therapeutic coating can be applied to and/or used in conjunction with other medical devices, such as, but not limited to, embolic filters, implantable devices, treatment devices, balloons, stents, etc.

Unlike conventional anticoagulants such as heparin and warfarin which inhibit various cofactors in the internal and external clotting cascade, and which may contribute to the serious systemic negative effects, the category of anticoagulants known as direct oral anticoagulants (DOACs) bind directly to specific clotting factors. Examples of DOACs include apixaban, rivaroxaban, edoxaban, dabigatran, betrixaban, and argatroban, which directly bind to factor Xa, and dabigatran, which directly binds to factor IIa (thrombin). The medical devices disclosed herein provide a way of achieving localized release of these DOACs at the surface of the device.

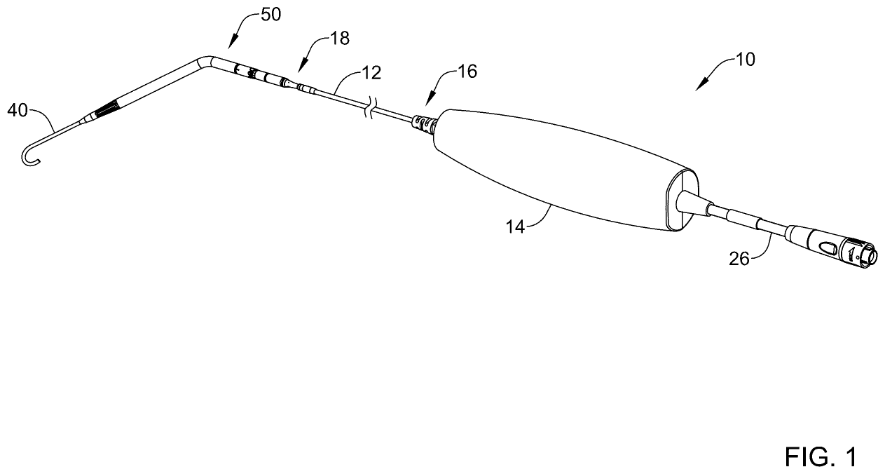

FIG. 1 illustrates a perspective view of a percutaneous circulatory support device 10 including a percutaneous blood pump 50 located at a distal end region thereof. The percutaneous circulatory support device 10 may be coupled to or include the blood pump 50, with an elongate shaft 12 of the percutaneous circulatory support device 10 extending proximally from the percutaneous blood pump 50 and a distal tip 40 extending distally from the blood pump 50. For instance, a proximal end 16 of the elongate shaft 12 may be coupled to a junction housing 14 and a distal end 18 of the elongate shaft 12 may be coupled to the percutaneous blood pump 50. The percutaneous circulatory support device 10 may also include an extension 26 connectable to the controller for sending and/or receiving signals, such as from one or more sensors during operation of the blood pump 50. In some cases, the extension may transfer current to the blood pump 50 and/or electrical and/or optical signals from diagnostic sensors (such as, but not limited to, blood pressure, blood flow velocity, or the like).

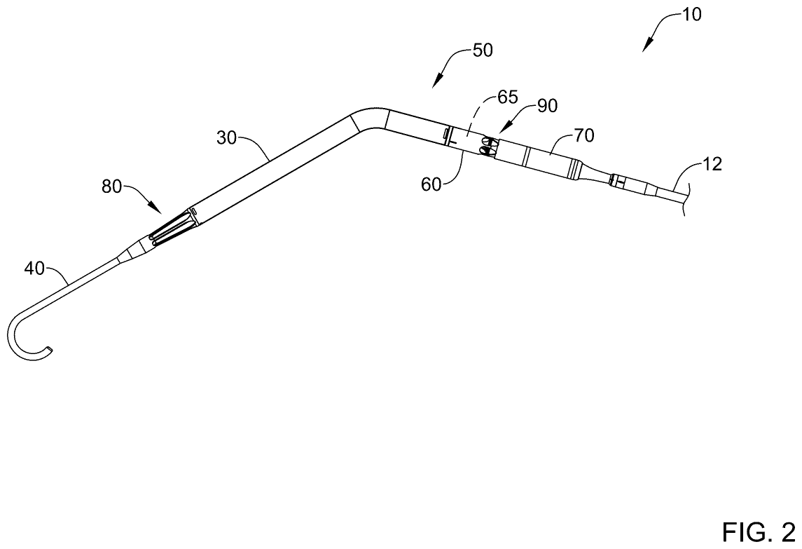

Additional features of the blood pump 50 are illustrated in FIG. 2. The blood pump 50 may generally include a flexible cannula 30, an impeller housing 60, and a motor housing 70. In some embodiments, the flexible cannula 30, the impeller housing 60 and/or the motor housing 70 may be integrally or monolithically constructed. In other instances, the flexible cannula 30, the impeller housing 60, and/or the motor housing 70 may be separate components. The impeller housing 60 carries an impeller assembly 65 therein. The impeller assembly 65 may include an impeller, secured to an impeller shaft, that rotates relative to the impeller housing 60 to drive blood through the blood pump 50. In some embodiments, the impeller shaft and the impeller of the impeller assembly 65 may be integrally formed, whereas, in other embodiments the impeller shaft and the impeller may be separate components.

Rotation of the impeller causes blood to flow from a blood inlet 80 of the blood pump 50, such as at a distal end of the flexible cannula 30, through the flexible cannula 30 and the impeller housing 60, and out of a blood outlet 90 proximal of the impeller, such as through a sidewall formed on the impeller housing 60. In some instances, the blood inlet 80 may include a plurality of blood inlet windows arranged around a circumference of the blood pump 50 (e.g., the flexible cannula 30). In some instances, the blood outlet 90 may include a plurality of blood outflow windows arranged around a circumference of the impeller housing 60. In other embodiments, the inlet 80 and/or the outlet 90 may be formed on other portions of the blood pump 50.

With continued reference to FIG. 2, the motor housing 70 carries a motor configured to rotatably drive the impeller of the impeller assembly 65 relative to the impeller housing 60. Electrical power may be supplied to the motor through wiring extending through the elongate shaft 12, for example. In some instances, the motor may be physically connected to the impeller. For example, in some embodiments the impeller may be mounted on the drive shaft of the motor. In other embodiments, the impeller shaft may be directly or indirectly coupled to the drive shaft of the motor. In some instances, the drive assembly may include a magnetic coupling between the motor and the impeller. For example, a driving magnet may be mounted on the drive shaft of the motor. Rotation of the driving magnet causes rotation of a driven magnet, which is connected to the impeller assembly 65. More specifically, in embodiments incorporating an impeller shaft, the impeller shaft and the impeller of the impeller assembly 65 are configured to rotate with the driven magnet. In other embodiments, the motor may be coupled to the impeller assembly 65 via other components.

The blood pump 50 may be guided over a guidewire during introduction of the blood pump 50 into the vasculature of a patient. For instance, a guidewire, inserted through a guidewire lumen of the distal tip 40, may be advanced proximally along the impeller assembly 65 and out through one of the outflow windows of the blood outlet 90. With the guidewire tracked through the blood pump 50, the percutaneous circulatory support device 10 may be advanced over the guidewire into a vasculature.

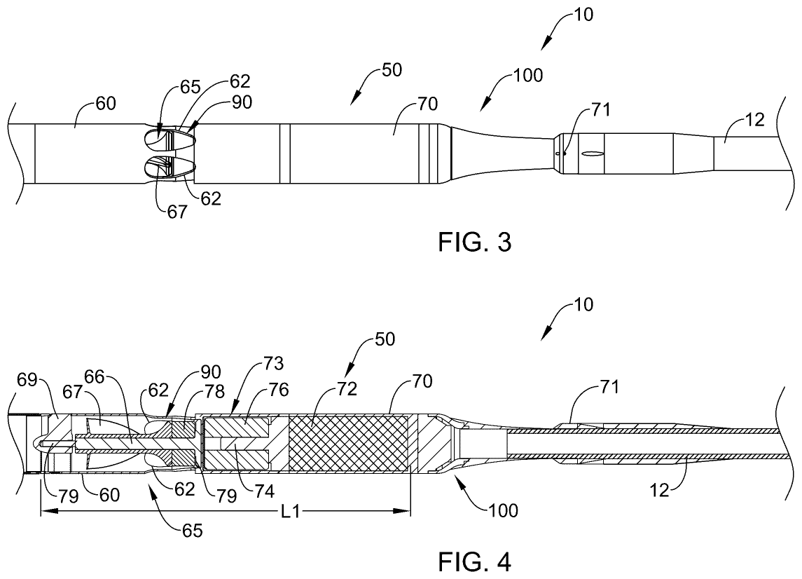

FIG. 3 is a side view of a portion of the percutaneous circulatory support device 10 illustrating a portion of the percutaneous blood pump 50 connected to the elongate shaft 12 at a junction 100. A proximal end of the impeller assembly 65 is also visible through the outflow windows of the blood outlet 90. FIG. 4 is a cross-sectional view of the portion of the percutaneous circulatory support device 10 of FIG. 3, including the junction 100 between the percutaneous blood pump 50 and the elongate shaft 12 of the percutaneous circulatory support device 10. The junction 100 may be configured to mechanically connect the motor housing 70 (e.g., a metallic motor housing) of the blood pump 50, or other component of the blood pump 50, to the distal end region of the elongate shaft 12 (e.g., a polymeric tubular member).

As shown in FIG. 4, the impeller assembly 65 includes a drive shaft 66 and an impeller 67 coupled thereto, where the drive shaft 66 is configured to rotate with the impeller 67. As shown, the drive shaft 66 is at least partially disposed within the impeller 67. A distal bearing support 69 is located distal of the distal end of the impeller assembly 65 and holds the drive shaft 66 concentric within the impeller housing 60. The impeller assembly 65 further includes a driven magnet 78 coupled to, and at least partially surrounding, the drive shaft 66 and/or the impeller 67. The driven magnet 78 may be any type of magnetic rotor capable of being driven by a driving magnet assembly. In this manner, as a magnetic field is applied to the driven magnet 78 by the driving magnet assembly, the driven magnet 78 rotates, causing the drive shaft 66 and impeller 67 to rotate.

As shown in FIG. 4, the driving magnet assembly includes a driving magnet 76 coupled to a drive line 74 configured to transfer torque from the motor 72 to the driving magnet 76. The motor housing 70 may be configured to hermetically seal the driving magnet 76 and the motor within the motor housing 70, and thus fluidly isolate the driving magnet 76 from the driven magnet 78. Electrical wires, not shown, may extend through a lumen of the elongate shaft 12 to provide electrical power to the motor 72. In other instances, the drive line 74 may be directly coupled to the impeller 67 and/or drive shaft 66, or other driven trains may be provided to transfer torque from the motor 72 to the impeller 67.

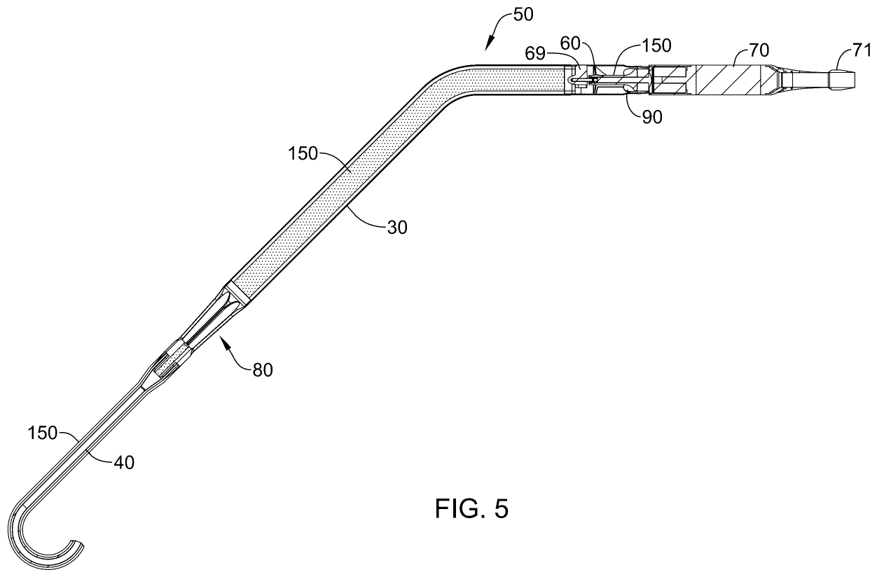

FIG. 5 is cross-sectional view of a portion of the percutaneous circulatory support device 10 of FIG. 2. The percutaneous circulatory support device 10 may include a drug eluting coating or therapeutic coating 150 disposed on one or more surfaces thereof. In some cases, the drug coating or coating composition 150 may include an anticoagulation drug, and more particularly a factor Xa inhibitor. The drug eluting coating 150 may be disposed along one or more regions of the percutaneous circulatory support device 10. In some examples, the drug eluting coating 150 may be disposed on an inner (e.g., luminal) and/or outer surface of the flexible cannula 30, an inner (e.g., luminal) and/or outer surface of the cannula 30 adjacent to the blood inlet 80, an outer and/or inner surface of the distal tip 40, a surface of the impeller housing 60, adjacent the blood outlet 90, the distal bearing support 69, etc. The drug eluting coating 150 may be an internal layer of the flexible cannula 30. For example, a slotted hypotube may form an inner layer or inner portion of the flexible cannula 30. The drug eluting coating 150 may be deposited on the outer diameter or outer surface of the hypotube. The hypotube can then be covered with a polyurethane sheath, or other polymeric sheath, that is melt bonded to the drug eluting coating 150 such that the polyurethane sheath forms an outer surface of the flexible cannula 30 and the hypotube and drug eluting coating 150 are disposed radially inwards of the polyurethane sheath. Said differently, the drug eluting coating 150 may be disposed between the slotted hypotube and the polyurethane sheath. During clinical use the drug can elute through the slots in the hypotube to the inner diameter or lumen of the cannula 30. In some cases, the drug eluting coating 150 may be deposited directly on different components of the percutaneous circulatory support device 10 that are at high risk of thrombus formation, such as, but not limited to, sensor ports 71, bearing surfaces 79, and the like. In some examples, the drug eluting coating 150 may be deposited on surfaces adjacent to the component that are at high risk of thrombus formation. However, the rotational components of the motor may be free from the drug eluting coating 150.

The drug eluting coating 150 may be applied to upstream components with enough dose that as the therapeutic agent elutes into the bloodstream, downstream pump components are protected from thrombus for the duration of the use of the percutaneous circulatory support device 10. As blood enters the interior of the flexible cannula 30 through the blood inlet 80, some of the therapeutic agent or drug within the drug eluting coating 150 may elute into the blood. As the blood travels through the interior of the cannula 30, over the impeller assembly 65, and out the blood outlet, the drug may be present within the blood at a therapeutic level to prevent thrombus formation on or at: the inlet 80, the inner surface of the flexible cannula 30, the outlet 90, the impeller assembly 65, the driven magnet 78, etc. Preventing the formation of thrombus may improve patient safety as well as improve pump functionality. It is contemplated that the percutaneous circulatory support device 10 may be protected from thrombus formation without the use of a systemic dose of anticoagulant. It is further contemplated that the drug eluting coating 150 may enable use of the percutaneous circulatory support device 10 for longer durations without a purge line while improving system simplicity and usability.

The drug eluting coating 150 may be disposed over an entirety of one or more surfaces or over portions thereof. For example, the drug eluting coating 150 may be disposed along only portions of the inner surface of the flexible cannula 30. This is just one example. The drug eluting coating 150 may be applied in any configuration or over any combination of surfaces desired. The drug eluting coating 150 disposed on the percutaneous circulatory support device 10 may have an average thickness in the range of about 1 micrometer (μm) to about 50 μm, for example.

The terms “therapeutic agents,” “drugs,” “bioactive agents,” “pharmaceuticals,” “pharmaceutically active agents”, and other related terms may be used interchangeably herein and include genetic therapeutic agents, non-genetic therapeutic agents, and cells. Therapeutic agents may be used singly or in combination. A wide range of therapeutic agent loadings can be used in conjunction with the devices of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

In some examples, the drug eluting coating 150 may include an anticoagulation drug such as, but not limited to, a factor Xa inhibitor. The drug eluting coating 150 may include a therapeutic agent that includes a DOAC, such as, but not limited to, apixaban, rivaroxaban, edoxaban, dabigatran, betrixaban, letaxaban, eribaxaban, and argatroban. However, other beneficial therapeutic agents may include, but are not limited to, anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

More specific drugs or therapeutic agents include antistasin, paclitaxel, rapamycin, sirolimus, everolimus, tacrolimus, heparin, diclofenac, aspirin, Epo D, dexamethasone, estradiol, halofuginone, cilostazol, geldanamycin, ABT-578 (Abbott Laboratories), trapidil, liprostin, actinomycin D, Resten-NG, Ap-17, abciximab, clopidogrel, Ridogrel, beta-blockers, bARKct inhibitors, phospholamban inhibitors, and SERCA 2 gene/protein, resiquimod, imiquimod (as well as other imidazoquinoline immune response modifiers), human apolipoproteins (e.g., AI, AII, AIII, AIV, AV, etc.), vascular endothelial growth factors (e.g., VEGF-2), as well as derivatives of the forgoing, among many others, and/or combinations thereof. Other drugs may include anti-inflammatory agents such as dexamethasone, prednisolone, corticosterone, budesonide, estrogen, sulfasalazine, mesalamine, and analogues thereof; antineoplastic/antiproliferative/anti-miotic agents such as paclitaxel, 5-fluorouracil, cisplatin, vinblastine, vincristine, epothilones, endostatin, angiostatin, thymidine kinase inhibitors, and analogues thereof; anesthetic agents such as lidocaine, bupivacaine, ropivacaine, and analogues thereof; anti-coagulants; and growth factors.

In some instances, a percutaneous circulatory support device 10 including a drug eluting coating 150 may provide a localized sustained delivery of a direct anti-coagulant which may reduce the risk of thrombus formation on the percutaneous circulatory support device 10. For example, the percutaneous circulatory support device 10 including a drug eluting coating 150 described herein uses direct oral anticoagulants (DOACs) (which are typically taken orally and used systemically) and provides them only on the surface of the device where they are needed, thus providing the advantage of reducing the potential adverse effects of systemic OAC therapy. A percutaneous circulatory support device 10 with a drug eluting coating 150 may be used to reduce the risk of thrombus formation on the percutaneous circulatory support device 10.

In some cases, rivaroxaban may be the drug used. Rivaroxaban, which is also known as (S)-5-Chloro-N-((2-oxo-3-(4-(3-oxomorpholino)phenyl)oxazolidin-5-yl)methyl)thiophene-2-carboxamide, has the following chemical structure:

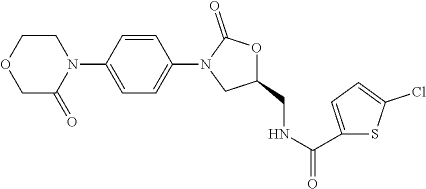 [SMILES: O=C(NC[C@H]1CN(c2ccc(N3CCOCC3=O)cc2)C(=O)O1)c1ccc(Cl)s1]

In some instances, the drug eluting coating 150 may include individual drug particles that are encapsulated with one or more excipients. The drug particles may include crystals of the drug, for example. Drug crystals may be formed in a variety of ways, for example. In some cases, a drug or other therapeutic agent may be available in an amorphous form, and a variety of processes may be used to convert an amorphous drug or other therapeutic agent into a crystalline drug or other therapeutic agent. In other examples, the therapeutic agent may be available in crystalline form. In yet other examples, the therapeutic agent may become crystalline during the process of making and applying the drug eluting coating 150. However, in some cases, the individual drug particles need not be encapsulated.

As described herein, the percutaneous circulatory support device 10, or portions thereof, may be coated with a drug eluting coating 150. As an example, the therapeutic composition or drug eluting coating 150 may include one or more therapeutic agents, such as, but not limited to, an anticoagulant such as rivaroxaban. Rivaroxaban crystals may be mixed with, dispersed in, and/or coated with an excipient or a mixture of excipients. Excipients can be used to enhance the durability of the drug eluting coating 150, facilitate drug transfer to the treatment location, and/or control drug dissolution. In some embodiments, one or more additional therapeutic agents may be provided in addition to rivaroxaban. In some cases, the one or more additional therapeutic agents may be added to the therapeutic composition. In other examples, the one or more additional therapeutic agents may be provided in a separate layer as an additional separate therapeutic composition. In some instances, the percutaneous circulatory support device 10, or portions thereof, may be contacted with the coating composition in order to form a coating on the medical device. In some instances, the percutaneous circulatory support device 10, or a portion thereof, may be dipped into the coating composition. In some cases, vapor deposition may be used to transfer the coating composition to the percutaneous circulatory support device 10. In some cases, a roller coating process may be used to transfer the coating composition to the percutaneous circulatory support device 10. In some cases, the coating composition may be sprayed onto the percutaneous circulatory support device 10 or may be sprayed onto a particular portion or region of the percutaneous circulatory support device 10. These are just some examples.

In addition to the therapeutic agent, the drug eluting coating 150 may further include one or more excipients. Excipients can be used to enhance the durability of the drug eluting coating 150, facilitate drug transfer to a target region, and/or control drug dissolution. It is contemplated that different excipients may be used depending on the use of the drug eluting coating. For example, it may be desirable for the drug eluting coating 150 to remain on the surface(s) of the percutaneous circulatory support device 10 while in other uses it may be desirable to transfer the drug eluting coating 150 to an adjacent body tissue. In some cases, the excipient may be flexible to bend with the flexible cannula 30. For example, the excipient may be a same material as the flexible cannula 30 or may have a flexibility similar to the flexible cannula 30.

An example excipient may include a thermoplastic polyurethane, such as, but not limited to, Pellethane® thermoplastic polyurethanes (TPUs). Pellethane® TPUs may be available as aromatic polyether and polyesters having a range of flexibilities and hardness. Other examples of thermoplastic polyurethanes are Tecoflex™ SG, which are a series of polyurethanes that are readily soluble in organic solvents. Other example excipients may include poly(vinylidene fluoride) (PVDF), polyvinylpyrrolidone (PVP), poly(vinylidene fluoride)-co-hexafluoropropylene (PVDF-HFP), polylactic acid (PLA). Yet other example excipients may include, but are not limited to, acetyl tri-butyl citrate (ATBC), acetyl tri-butyl citrate (ATHC), poly-DL-lactide (PDLLA), poly-L-lactide (PLLA), poly-D-lactide (PDLA), poly(lactic-co-glycolic acid) (PLGA), etc. It is contemplated that the release rate of rivaroxaban can be increased or decreased based on the properties of the excipient, such as, but not limited, a porosity of the excipient, a degradation rate of the excipient (if degradable), a rate of water diffusion into the excipient, etc. In one example, PLGA may degrade faster than PLA. By using a faster degrading polylactide/glycolide copolymer (PLGA) the release rate of the therapeutic agent can be increased. The release from PLGA can be controlled by controlling the lactide to glycolide ratio and polymer molecular weight.

In some examples, additional components may also be used in the drug eluting coating 150. In some cases, an antioxidant, such as, but not limited to, butylated hydroxytoluene (BHT) may be used in the composition.

In an illustrative example, the drug eluting coating 150 may include a hemocompatible polymer such as, but not limited to, a thermoplastic polyurethane (TPU) and a therapeutic agent such as an anticoagulant, and in some cases, a direct oral anticoagulant (DOAC). The therapeutic agent may form crystalline particles within a continuous TPU matrix. The resulting drug eluting coating 150 may be applied to one or more surfaces of the percutaneous circulatory support device 10 to act as a drug depot for sustained localized release.

It is contemplated that the surface area of the percutaneous circulatory support device 10 over which the drug eluting coating 150 is disposed and/or a time period over which the percutaneous circulatory support device 10 is in situ may determine, at least in part, how long the drug eluting coating 150 releases the drug. In some cases, the surface area of the drug eluting coating 150 may be adjusted to provide the desired duration of drug release. For example, the drug eluting coating 150 may be applied to a greater surface area of the percutaneous circulatory support device 10 and thus increase the volume of drug eluting coating 150 (and thus the drug) that is placed on the percutaneous circulatory support device 10. In some examples, the percutaneous circulatory support device 10 may be sized and shaped to provide the ability to build in drug reservoirs to provide long duration (e.g., about 1 year) release of DOAC and other drugs to locally reduce thrombus formation

In one embodiment, a polymer coating such as TPU, and one or more DOACs are dissolved in a solvent suitable for dissolving the polymer and drug. In some examples, the solvent may be a blend of acetone and N,N-dimethyl formamide (DMF). However, other solvents such as, but not limited to, N,N-dimethyl acetamide (DMAc), dimethylsulfoxide (DMSO), tetrahydrofuran (THF), N-methyl pyrrolidone (NMP), or the like may be used. This solution (e.g., TPU, DOAC, and solvent) may be applied directly to the percutaneous circulatory support device 10 by a dip coating or spray process. It is contemplated that less than an entirety of the percutaneous circulatory support device 10 may be coated with the drug eluting coating 150. A surface of the percutaneous circulatory support device 10 may be treated prior to applying the drug eluting coating 150 to improve the adhesion of the drug eluting coating 150 to the percutaneous circulatory support device 10. Some illustrative surface treatments may include, but are not limited to, plasma treating, silane coupling agents, surface roughening, and the like.

The drug eluting coating 150 may be applied to achieve a coating density of between 1-40 micrograms (μg) drug per square millimeter (mm) (μg/mm²) on one or more surfaces of the percutaneous circulatory support device 10. The drug eluting coating 150 may include in the range of about 5 weight percent to about 45 weight percent therapeutic agent and in the range of about 55 weight percent to about 95 weight percent excipient (e.g., TPU). The weight ratio of therapeutic agent to excipient may be varied to achieve a desired drug release profile. In some examples, the drug eluting coating 150 may include in the range of about 30 weight percent to about 50 weight percent therapeutic agent and in the range of about 50 weight percent to about 70 weight percent excipient (e.g., TPU). The drug eluting coating 150 on the percutaneous circulatory support device 10 may have a thickness of up to 10 μm or more and an anticoagulant content (therapeutic agent content) in the range of about 1-40 μg/mm² may be achievable on the percutaneous circulatory support device 10. It is contemplated that a drug eluting coating 150 including TPU and a DOAC, such as rivaroxaban, may release in the range of about 40-80 percent of the therapeutic agent in about 7-60 days when the percutaneous circulatory support device 10 is in situ.

In a further example, a DOAC, such as rivaroxaban, may be dissolved in a solvent and a polymer excipient. The subsequent mixture may then then be deposited on the percutaneous circulatory support device 10 by spray or dip coating. A top layer of a polymer that contains no drug may be deposited over the drug eluting coating 150 to further modulate the drug release rate. In some examples, the polymer topcoat may include TPU. It is contemplated that increasing the weight percent of the polymer topcoat relative to the weight of the drug eluting coating 150 may slow a release of the therapeutic agent. More than one layer of polymer may be disposed over the drug eluting coating 150, as desired.

It is contemplated that any of the drug coating compositions described herein may be disposed over a primer layer. The primer layer may be applied directly to the percutaneous circulatory support device 10 prior to applying the drug eluting coating 150. The primer layer may be applied to the percutaneous circulatory support device 10 using coating processes, such as, spray coating, dip coating, roller coating, vapor deposition, and/or the like, and/or other suitable coating processes. In some examples, the primer layer may be a polymer dissolved in a solvent or mixture of solvents. An illustrative primer layer for the percutaneous circulatory support device 10 may include poly(butyl methacrylate) (PBMA) (2 percent solids) dissolved in a solvent mixture that includes in the range of about 70 percent acetone and 30 percent cyclohexanone.

### Process for Forming Crystalline Rivaroxaban

Crystalline rivaroxaban may be generated by crystallization of the rivaroxaban from a solution of a mixture of N,N-dimethyl formamide (DMF) and water. Crystalline rivaroxaban may also be generated by slowly adding a solution of rivaroxaban in DMF (1 to 4 wt % solids) into a large excess of deionized (DI) water.

### Experimental Results

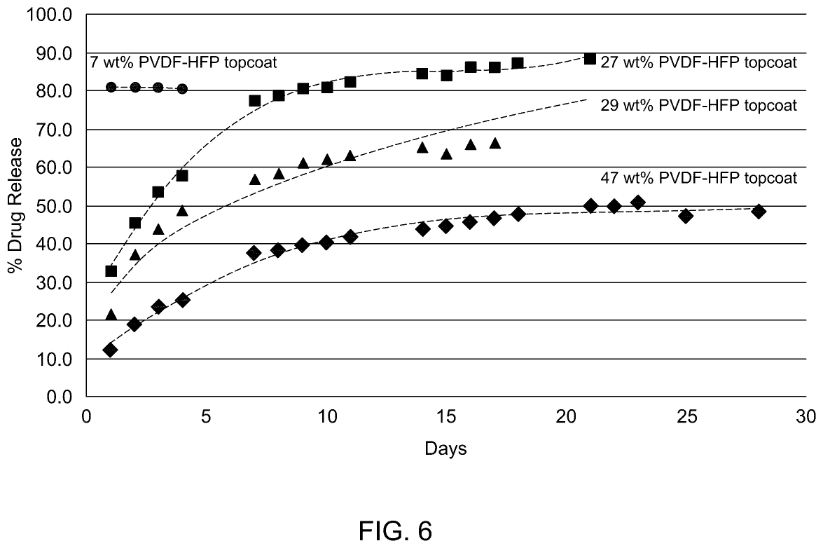

FIG. 6 is a graphical representation of experimental data providing relative drug release rates when stents were coated with a base coat of a drug coating including 60 weight percent PVDF-HFP and 40 weight percent rivaroxaban. Each drug coating was coated with a topcoat of PVDF-HFP having varying weight percents relative to the drug coating.

Innova™ self-expanding stents (8 mm×20 mm; commercially available from Boston Scientific®) were spray coated with several PVDF-HFP/rivaroxaban coating formulations. The coatings consisted of a base coat made up of 60/40 (wt/wt) PVDF-HFP/rivaroxaban. The base coat was sprayed from a 2% (wt/wt) solution of the polymer/drug in 60/40 acetone/DMF. The base coat was spray overcoated with PVDF-HFP at different thicknesses. As the stents were all the same size, increasing the thickness of the PVDF-HFP topcoat also increased the weight of the topcoat. The overcoat was sprayed from a solution of 2 wt % solids of PVDF-HFP in acetone. Table 1 below shows the formulations coated. In-vitro drug release from the coated stents was performed by incubating the stents in phosphate buffered saline (PBS) (pH˜7)/TWEEN® 20 solution at 37° C. and 120 rpm on an orbital shaker incubator. Drug elution was quantified by analyzing the phosphate buffered saline solution using high-performance liquid chromatography (HPLC) at various timepoints. The drug release curves are shown in FIG. 6.

TABLE 1
Coating formulations of drug coated stents.

| | | | | | Weight % |
|---|---|---|---|---|---|
| | Base Coat | | Topcoat | | Topcoat |
| | Weight | Base Coat | Weight | Topcoat | (topcoat to |
| Sample | (mg) | Composition | (mg) | Composition | basecoat) |
| 1 | 4.2 | 60 wt % | 0.31 | 100% | 7.2 |
| | | PVDF-HFP; 40 | | PVDF-HFP | |
| | | wt % | | | |
| | | rivaroxaban | | | |
| 2 | 4.1 | 60 wt % | 1.1 | 100% | 27 |
| | | PVDF-HFP; 40 | | PVDF-HFP | |
| | | wt % | | | |
| | | rivaroxaban | | | |
| 3 | 4.1 | 60 wt % | 1.2 | 100% | 29 |
| | | PVDF-HFP; 40 | | PVDF-HFP | |
| | | wt % | | | |
| | | rivaroxaban | | | |
| 4 | 4.2 | 60 wt % | 2.0 | 100% | 47 |
| | | PVDF-HFP; 40 | | PVDF-HFP | |
| | | wt % | | | |
| | | rivaroxaban | | | |

FIG. 6 illustrates how drug release can be controlled by varying the weight and thickness of PVDF-HFP topcoat. A range of drug release durations from approximately 1 day to more than 30 days are possible by tailoring the topcoat thickness and/or weight. For example, as can be seen in FIG. 6, increasing the weight percent of the topcoat, and thus the thickness, slowed the release of the rivaroxaban. For example, the sample having a topcoat of 7 weight percent PVDF-HFP released approximately 80% of the rivaroxaban within a day. The sample having a topcoat of 27 weight percent PVDF-HFP released approximately 80% of the rivaroxaban within about 9 days and 90% of the rivaroxaban within about 21 days. The sample having a topcoat of 29 weight percent PVDF-HFP released approximately 80% of the rivaroxaban within about 21 days. The sample having a topcoat of 47 weight percent PVDF-HFP released approximately 50% of the rivaroxaban within about 28 days.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A percutaneous circulatory support device comprising:
a flexible elongate shaft;
a percutaneous blood pump coupled to a distal end region of the flexible elongate shaft at a junction, the percutaneous blood pump configured to pump blood through a housing of the percutaneous blood pump during use; and
a drug eluting coating disposed on at least a portion of a surface of the percutaneous blood pump.

2. The percutaneous circulatory support device of claim 1, wherein the drug eluting coating comprises:
an excipient; and
a direct oral anticoagulant (DOAC).

3. The percutaneous circulatory support device of claim 2, wherein the excipient comprises a thermoplastic polyurethane.

4. The percutaneous circulatory support device of claim 2, wherein the excipient comprises in the range of about 60 to 95 weight percent of the drug eluting coating and the DOAC comprises in the range of about 5 to 40 weight percent of the drug eluting coating.

5. The percutaneous circulatory support device of claim 2, wherein the excipient comprises in the range of about 50 to 70 weight percent of the drug eluting coating and the DOAC comprises in the range of about 30 to 50 weight percent of the drug eluting coating.

6. The percutaneous circulatory support device of claim 1, further comprising a top layer disposed over the drug eluting coating.

7. The percutaneous circulatory support device of claim 6, wherein the top layer is free from a therapeutic agent.

8. The percutaneous circulatory support device of claim 2, wherein the DOAC is present in the drug eluting coating in an amount of between 1-40 micrograms per square millimeter.

9. The percutaneous circulatory support device of claim 1, wherein the percutaneous blood pump comprises:
a flexible cannula;
an impeller housing; and
a motor housing.

10. The percutaneous circulatory support device of claim 9, wherein the flexible cannula comprises a slotted hypotube covered with a polyurethane sheath.

11. The percutaneous circulatory support device of claim 10, wherein the drug eluting coating is disposed between the slotted hypotube and the polyurethane sheath.

12. The percutaneous circulatory support device of claim 9, wherein the drug eluting coating is disposed on an inner surface of the flexible cannula.

13. The percutaneous circulatory support device of claim 9, wherein the drug eluting coating is disposed on a portion of the impeller housing.

14. The percutaneous circulatory support device of claim 2, wherein the DOAC is apixaban, rivaroxaban, or edoxaban.

15. The percutaneous circulatory support device of claim 1, wherein the drug eluting coating has a thickness in the range of about 1 to 50 micrometers.

16. A percutaneous circulatory support device comprising:
a flexible elongate shaft;
a percutaneous blood pump coupled to a distal end region of the flexible elongate shaft at a junction, the percutaneous blood pump configured to pump blood through a housing of the percutaneous blood pump during use; and
a drug eluting coating disposed on at least a portion of a surface of the percutaneous blood pump, wherein the drug eluting coating comprises:
an excipient; and
an anticoagulant.

17. The percutaneous circulatory support device of claim 16, wherein the anticoagulant comprises a factor Xa inhibitor.

18. The percutaneous circulatory support device of claim 17, wherein the excipient comprises in the range of about 60 to 95 weight percent of the drug eluting coating and the anticoagulant comprises in the range of about 5 to 40 weight percent of the drug eluting coating.

19. The percutaneous circulatory support device of claim 16, further comprising a top layer disposed over the drug eluting coating.

20. A percutaneous circulatory support device comprising:
a flexible elongate shaft;
a percutaneous blood pump including a flexible cannula, an impeller housing, and a motor housing, the percutaneous blood pump coupled to a distal end region of the flexible elongate shaft at a junction and configured to pump blood through a housing of the percutaneous blood pump during use; and
a drug eluting coating disposed on at least a portion of a surface of the percutaneous blood pump, wherein the drug eluting coating comprises:
an excipient; and
an anticoagulant.
